(19) United States
(12) Patent Application Publication
(10) Pub. No.: US 2026/0272582 A1
(43) Pub. Date: Sep. 17, 2026

(54) ROBOTIC TELESURGERY PORTAL SYSTEM AND METHOD

(71) Applicant: SOFTACUITY INC., Santa Barbara, CA (US)

(72) Inventors: Bruce Laurence Kennedy, Santa Barbara, CA (US); Matthew Wood, Goleta, CA (US)

(21) Appl. No.: 19/167,794

(22) PCT Filed: Mar. 22, 2024
(86) PCT No.: PCT/US2024/021202

§ 371 Date: Sep. 22, 2025

Related U.S. Application Data
(60) Provisional application No. 63/454,262, filed on Mar. 23, 2023.

(51) Int. Cl. A61B 34/35; A61B 90/00; G16H 40/67

(52) U.S. Cl. CPC A61B 34/35; A61B 90/361; G16H 40/67; A61B 2090/373

(57) ABSTRACT

A robotic telesurgery portal system has a first robotic telesurgery portal at a first location, the first portal being couplable to a surgical robot and configured to: receive surgical video and robot control feedback data from the surgical robot; synchronize the video and robot control feedback data; transmit the surgical video and robot control feedback data; receive robot control information; and communicate robot control information to the surgical robot. The system also has a second robotic telesurgery portal at a second location, the second portal being couplable to a surgical robot control console and configured to: receive surgical video and robot control feedback data from the first robotic telesurgery portal; communicate the received surgical video and robot control feedback data to the surgical robot control console; receive robot control data; and transmit the received robot control data to the first robotic telesurgery portal.

### BACKGROUND

The present disclosure relates to devices used in surgery and, more particularly, to a system for enabling robotic telesurgery.

Robot-assisted surgery has allowed for tremendous surgical improvements. Typically, a surgical robot includes a camera and mechanical arms. Surgical tools are placed in the mechanical arms and controlled by a surgeon using a surgical robot control console. The surgical robot control console gives the surgeon a view of the surgical site and allows the surgeon very precise control of the mechanical arms. This allows for minimally invasive surgery and has led to surgeries with fewer complications and less scarring.

However, the surgical robot control console is typically positioned very close to the operating table with the patient. Accordingly, robot-assisted surgery has been primarily limited to situations where the surgeon is located with the surgical robot and the patient. This limits the applicability of robot-assisted surgery to situations where a surgeon with the specific skills required for the surgery can be located with the patient.

There is therefore a need for a system and method that allows surgeons to perform robot-assisted surgery from a location remote from the surgical robot and the patient. This would allow for the most skilled surgeons for a particular surgery to treat patients in distant locations.

### SUMMARY

The present application is directed to a robot telesurgery portal system that provides safe and effective visualization of surgical video, ensured routing of low-latency telesurgery robotic control, feedback, and video signals. The robot telesurgery portal system also provides telepresence. between a patient-side portal (PsP) and a surgeon-side portal (SsP).

In an implementation, a robotic telesurgery portal system has a first robotic telesurgery portal at a first location, the first robotic telesurgery portal being couplable to a surgical robot and configured to: receive surgical video and robot control feedback data from the surgical robot; synchronize the video and robot control data from the surgical robot; transmit the surgical video and robot control feedback data; receive robot control information; and communicate robot control information to the surgical robot. The robotic telesurgery portal system also has a second robotic telesurgery portal at a second location remote from the first location, the second robotic telesurgery portal being couplable to a surgical robot control console and configured to: receive surgical video and robot control feedback data from the first robotic telesurgery portal; communicate the received surgical video and robot control feedback data to the surgical robot control console; receive robot control data from the surgical robot control console; and transmit the received robot control data to the first robotic telesurgery portal.

The first robotic telesurgery portal and the second robotic telesurgery portal are configured for multiple low-latency modes of secure and redundant communication. In an implementation, at least one of the low-latency modes of communication is through a 5G cellular communication network. Optionally, the first robotic telesurgery portal and the second robotic telesurgery portal are configured to prevent the commencement of a surgery until multiple modes of secure and redundant communication are verified.

In an implementation, the first robotic telesurgery portal is further configured to synchronize surgical video and robot control feedback data prior to transmitting the surgical video and robot control feedback data. The first robotic telesurgery portal may be further configured to encode the surgical video and robot control feedback data prior to transmitting the surgical video and robot control feedback data. The first robotic telesurgery portal may be further configured to variably encode the surgical video and robot control feedback data depending on sensed communication bandwidth to ensure that sufficient surgical video and robot control feedback data is communicated to the second robotic telesurgery portal.

In an implementation, each of the first robotic telesurgery portal and the second robotic telesurgery portal further comprise: a microphone; a speaker; a camera; and a display; and wherein each of the first robotic telesurgery portal and the second robotic telesurgery portal are further configured to communicate telepresence data received from the microphones and cameras to the speakers and displays. A single robotic telesurgery portal may be configurable as both the first robotic telesurgery portal and the second robotic telesurgery portal.

These and other features are described below.

### BRIEF DESCRIPTION OF THE DRAWINGS

The features, aspects and advantages of the present invention will become better understood with regard to the following description, appended claims and accompanying figures wherein:

FIG. 1 is a schematic diagram of a robotic telesurgery portal system located at a patient site with a surgical robot according to an implementation;

FIG. 2 is a schematic diagram of a robotic telesurgery portal system located remote from the patient site at a surgeon site with a surgical robot control console according to an implementation; and

FIG. 3 is a schematic diagram of a system having robotic telesurgery portal systems according to an implementation.

### DETAILED DESCRIPTION

In the following description of the preferred implementations, reference is made to the accompanying drawings which show by way of illustration specific implementations in which the invention may be practiced. Wherever possible, the same reference numbers will be used throughout the drawings to refer to the same or like parts. It is to be understood that other implementations may be utilized and structural and functional changes may be made without departing from the scope of this disclosure.

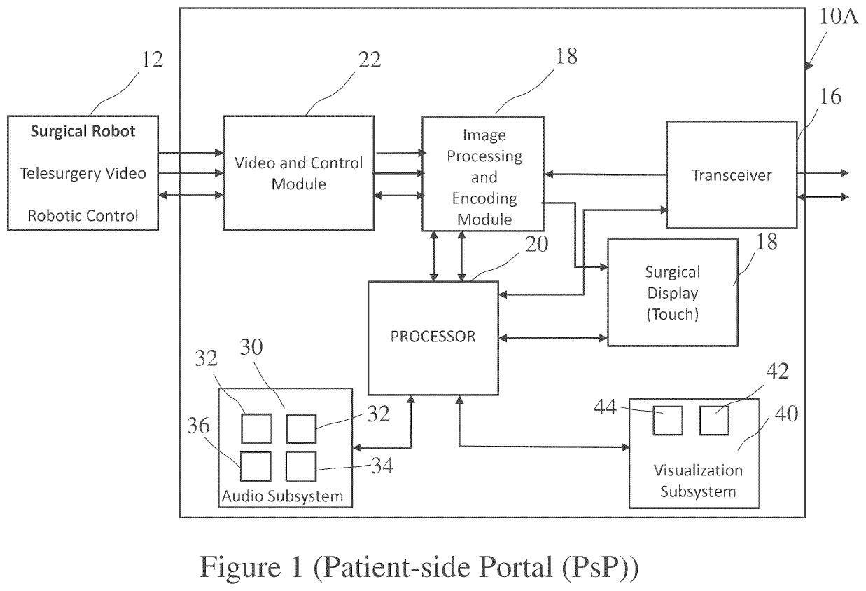

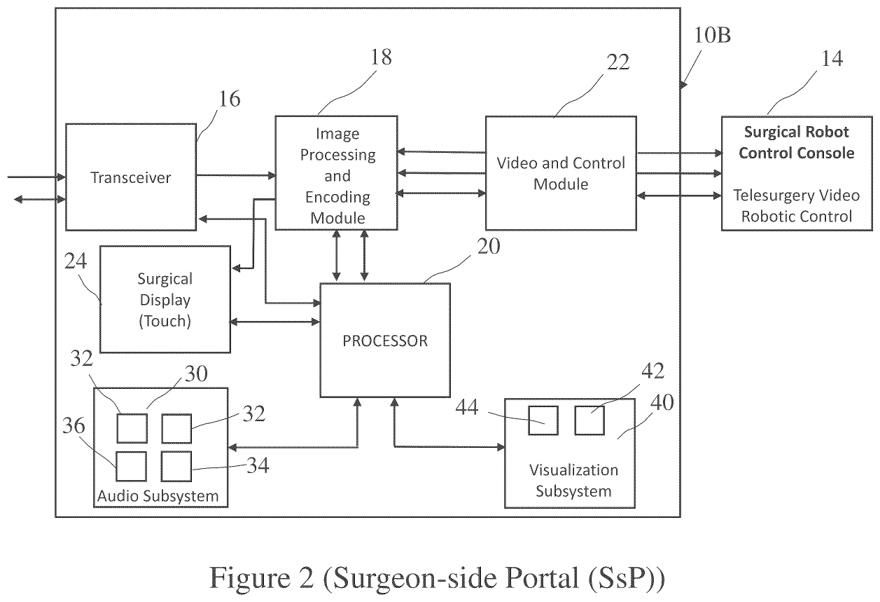

With reference to FIGS. 1 and 2 a robotic telesurgery portal system according to various implementations is described below. In an implementation, as shown in FIG. 1, a first robotic telesurgery portal 10A is located in a patient's operating room with a surgical robot 12. The first robotic telesurgery portal 10A is sometimes referred to herein as a “patient-side portal (PsP).” As shown in FIG. 2, a second robotic telesurgery portal 10B is located remote from the patient site at a surgeon site with a surgical robot control console 14. The second robotic telesurgery portal 10B is sometimes referred to herein as a “surgeon-side portal (SsP).” In an implementation, the first robotic telesurgery portal 10A and the second robotic telesurgery portal 10B are each connectable to a surgical robot and a robotic telesurgery portal and are substantially interchangeable. In an additional implementation, the first robotic telesurgery portal 10A is configured for use with a specific surgical robot and the second robotic telesurgery portal 10B is configured for use with a specific surgical robot control console.

Each robotic telesurgery portal 10A and 10B has a transceiver 16 for communicating with another robotic telesurgery portal. The transceiver 16 is configured for multiple modes of secure communication. For example and without limitation, the transceiver 16 may be configured for 5G cellular communication. Additionally, the transceiver 16 may be configured for wired and wireless broadband wide area network (WAN) communication. In an implementation, the transceiver 16 is configured for wired communication over private wide area network lines to ensure low latency transmission. The transceiver is further configured for redundant communication so that the same data is transferred over multiple modes. A cellular interface and redundant communications are particularly important in the robotic telesurgery context to ensure communication of critical information in an emergency if a wide area network connection fails. In an implementation, the robotic telesurgery portal 10A, 10B contains two transceivers 16 for increased bandwidth and in case of a hardware failure.

The transceiver 16 is coupled to an image processing module (IPM) 18 and a processor 20. The IPM 18 may include a field programmable gate array (FPGA). The IPM 18 is coupled to the processor 20. The IPM 18 is also coupled to a telesurgery video and control input/output module 22. In an implementation, the telesurgery video and control input/output module 22 is connectable to the surgical robot 12 or the surgical robot control console 14. As explained below, the IPM 18 is responsible for encoding and decoding video.

In an implementation, the telesurgery video and control module 22 is multi-standard and connectable to multiple different types of surgical robots 12. In an additional implementation, the video and control module 22 is configured for connection to a specific surgical robot 12. In an implementation, the video and control module 22 is multi-standard and connectable to multiple different types of surgical robot control consoles 14. In an additional implementation, the video and control module 22 is configured for a connection to a specific robot control console 14.

In implementations, the video and control module 22 receives and transmits 1080 p video through a 3G-Serial Digital Interface (3G-SDI), Digital Visual Interface (DVI), or High Definition Multimedia Interface (HDMI). In implementations, the video and control module 22 receives ultra high definition video or 4K video through a High Definition Multimedia Interface (HDMI), 12G-Serial Digital Interface (12G-SDI), or a Display Port Interface. In implementations, the video and control module 22 receives and transmits robot control and feedback using a Controller Area Network (CAN) bus, Ethernet for Control Automation Technology (EtherCat), Universal Serial Bus (USB) 3.0, or a Transmission Control Protocol/Internet Protocol (TCP/IP) Interface.

The processor 20 may be, for example, a Jetson Orin NX by Nvidia. A video signal may be passed from the IPM 18 to the processor 20. The processor 20 may be used to augment the video signal and return the video signal to the IPM 18. For example, the processor 20 may add overlays and prompts to the video. While the IPM 18 and the processor 20 are shown as separate units, they can be implemented as a single module.

The processor 20 is coupled to an audio subsystem 30 containing at least one microphone 32 and a visualization subsystem 40 containing at least one camera 42 such as for capturing images of an operating room. The audio subsystem 30 and the visualization subsystem 40 are used for telepresence, to enable to a surgeon to feel connected to the patient's operating room and to enable people in the patient's operating room feel connected to the surgeon, as discussed further below.

The audio subsystem 30 may contain an array of microphones 32. In an implementation, the audio subsystem 30 contains two microphones 32 configured for stereo. In an implementation, the audio subsystem 30 contains five microphones 32. The microphones 32 may be positioned at various points along an outer edge of the robotic telesurgery portal 10A, 10B. The microphones 32 may be configured to beam steer and cross noise cancel.

Additionally, the audio subsystem 30 may contain at least one speaker 34. Additionally, the audio subsystem 30 may contain a digital signal processor (DSP) 36 for managing audio detected by the at least one microphone 32 and for playing audio through the at least one speaker 34. While the digital signal processor 36 and the processor 20 are shown as separate units, they can be implemented in a single module. In an implementation, the audio subsystem 30 contains two speakers 34 and subwoofer for typical Dolby 2.1 audio 34. The robotic telesurgery portal 10A, 10B may also have indicator lights 38. The indicator lights 38 may display status, such as for example, the status of internet connectivity. The audio subsystem 30 can be used in conjunction with processor 20 to perform various functions, for example and without limitation for: voice control of the robotic telesurgery portal 10A, 10B, audio prompts to a user, user feedback, alarm notifications, alarm detection, telemedicine, communication, and for playing music.

The visualization subsystem 40 may have multiple cameras 42. In an implementation, the visualization subsystem 40 has stereo cameras 42 for assisting with room situational awareness or facial recognition. In an implementation, at least one camera 44 is a time of flight camera. In an implementation, the visualization subsystem 40 has two cameras 42 for stereo imaging. In an additional implementation, the visualization subsystem 40 has two cameras for three dimensional image and video capture. The visualization subsystem 40 can be used in conjunction with the processor 20 for viewing the surroundings of the robotic telesurgery portal 10A, 10B, such as an operating room. Additionally, the visualization subsystem 40 can be used in conjunction with the processor 20 to perform various function, for example and without limitation for: situational awareness, learning, training, telemedicine, and distance measurement.

The robotic telesurgery portal 10A, 10B may have a wireless network interface, such as Wi-Fi or Bluetooth, to couple the robotic telesurgery portal 10A, 10B to compatible external devices including, for example, input devices such as mice and keyboards, display devices, and portable computing devices such as phones, tablets and laptops for exchanging information.

Additionally, the robotic telesurgery portal 10A, 10B may have a secondary display interface for connection to an additional display. The additional display may be a touchscreen and may allow for annotation of images, records, or reports, or may be usable for control of the robotic telesurgery portal 10A, 10B. The secondary display interface may be, for example and without limitation, an HDMI, Display Port connector, or a Serial Digital Interface (SDI) connector. Additionally, the secondary display interface may include a universal serial bus (USB) interface for touchscreen control.

The robotic telesurgery portal 10A, 10B has numerous features. The robotic telesurgery portal 10A, 10B may be configured for authenticating users using at least one form of biometric authentication. In an implementation, the authentication system utilizes voice recognition as sensed by the at least one microphone 32 and at least partial face identification as sensed by the at least one camera 42. Once authenticated, a user may be provided access to hospital records and the ability to interact with other robotic telesurgery portals.

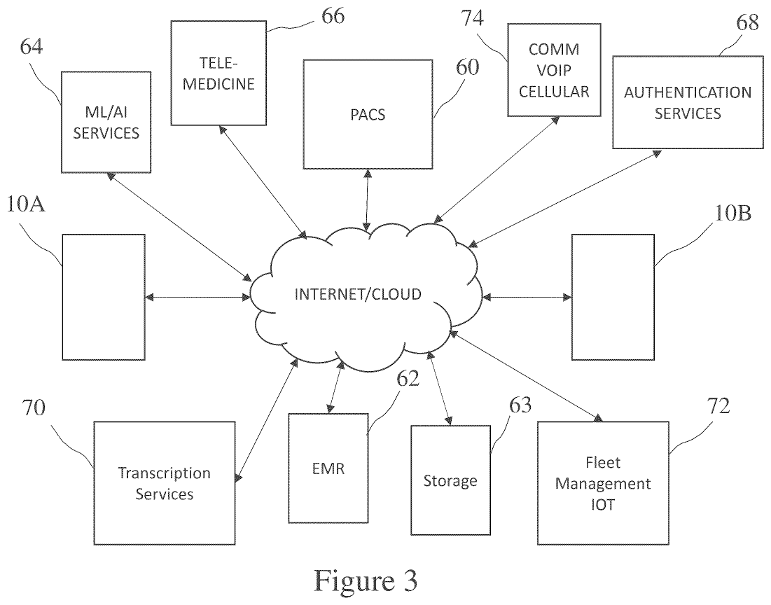

With reference to FIG. 3, the robotic telesurgery portal 10A, 10B may be linked to hospital networks, databases, and outside data processing. Once a user is authenticated, then information specific to that user, and to patients in the operating room, may automatically be gathered. For example, the robotic telesurgery portal 10A, 10B may be linked to a picture archiving and communication system (PACS) 60 either inside or outside of a hospital for obtaining or storing patient information. Additionally, the robotic telesurgery portal 10A, 10B may be linked to an electronic medical record (EMR)/electronic health record (HER) server 62 either inside or outside of a hospital for obtaining or storing patient information. Additionally, the robotic telesurgery portal 10A, 10B may be linked to a cloud based storage system 63.

Additionally, the robotic telesurgery portal 10A, 10B may be linked to external data processing systems and applications. The ability to link to external data storage and processing systems and applications allows for deeper artificial intelligence and machine learning functions. For example, if a voice command is received asking the robotic telesurgery portal 10A, 10B to “turn the lighting all the way up”, then the command may be processed by an external application and a command returned to the robotic telesurgery portal to turn the lighting to 100%. The robotic telesurgery portal 10A, 10B may be linked to cloud or local services via applications (apps) resident on the robotic telesurgery portal. The robotic telesurgery portal 10A, 10B may learn locally or through machine learning cloud services based on the events in the operating room and provide guidance about the events in the surgical suite to users and administrators via artificial intelligence.

Referring to FIG. 3, in an implementation, the robotic telesurgery portal 10A, 10B may be linked to machine learning and/or artificial intelligence applications and services 64. Additionally, the robotic telesurgery portal 10A, 10B may be linked to telemedicine services 66. Additionally, the robotic telesurgery portal 10A, 10B may be linked to external authentication services 68 as well as external transcription services 70 and external fleet management services 72. Additionally, the robotic telesurgery portal 10A, 10B may be linked to external communication services 74 such as voice over internet protocol (VOIP) and cellular communications.

Additionally, a user may also be able to control several different aspects of the operating room environment. The user may be able to exercise that control using one or more of: voice commands, gestures as sensed by at least on camera, built in user input devices, and user input devices coupled to the robotic telesurgery portal 10A, 10B. In an implementation, a touch screen surgical display 24 is coupled to the processor 20 such as by a universal serial bus (USB) interface 28. In an implementation, the surgical display 24 is also coupled to the IPM 18 for displaying a secure picture on the surgical display regardless of whether the processor is operational.

Using at least one camera, the robotic telesurgery portal 10A, 10B may be able to identify and track each of the people in the operating room as well as when each person enters and exits. This may include entry and exit of a patient. The robotic telesurgery portal 10A, 10B may also record video and audio of a surgical procedure as a record of the procedure, for producing reports, and for training purposes.

Operation of a system of robotic telesurgery portals 10A, 10B will now be further described. Surgical video and robot control feedback data from the surgical robot 12 enters the patient-side robotic telesurgery portal 10A through the video and control module 22. In an implementation, the surgical video is encoded by the IPM 18 to reduce required data bandwidth. In an implementation, the surgical video is encoded using an H.264 encoder. In a preferred implementation, the surgical video is encoded using an H.265 encoder. In an implementation, the encoder and decoder are implemented in the IPM 18 to minimize latency.

The surgical video is synchronized to the received robotic control feedback data by the IPM 18 and/or the processor 20. In an implementation, video and control data are synchronized using one or more of the following: a) time-stamping, b) a synchronization word associated with the packetized data before and after transmission; and c) a phase-locked loop on a receiving end.

The synchronized surgical video and robot control feedback data is transmitted by the transceiver 16 over secure redundant low-latency networks. For example, the synchronized surgical video and robot control feedback data may be transmitted over a 5G cellular data network and an ethernet connected wide area network. Additionally, the patient-side robotic telesurgery portal 10A device provides telepresence functionality by transmitting audio and video received from the audio subsystem 30 and the video subsystem 40.

The surgeon-side robotic telesurgery portal 10B receives the synchronized surgical video and robot control feedback data from the patient-side robotic telesurgery portal 10A through the transceiver 16. The received synchronized surgical video and robot control feedback data is routed through the IPM 18, decoded and then output through the video and control module 22 into the surgical robot control console 14 for visualization and observation by the surgeon. Additionally, telepresence information received from the patient-side robotic telesurgery portal 10A is communicated through the display 24 and audio subsystem 30 of the surgeon-side robotic telesurgery portal 10B.

Robot control signals generated by the surgeon operating the robotic control console 14 are routed into the surgeon-side robotic telesurgery portal 10B through the video and control module 22. Additionally, the surgeon-side robotic telesurgery portal 10B device provides telepresence functionality by transmitting audio and video received from the audio subsystem 30 and the video subsystem 40.

The robot control signals generated by the surgeon are encoded by the IPM 18 and transmitted over the secure, redundant, low-latency networks to the patient-side robotic telesurgery portal 10A. The robot control signals generated by the surgeon are received in the transceiver 16 of the patient-side robotic telesurgery portal 10A. The received robot control signals are routed through the IPM 18, decoded and then output through the video and control module 22 into the surgical robot 12 for implementation by the surgical robot. Additionally, telepresence information received from the surgeon-side robotic telesurgery portal 10B is communicated through the display 24 and audio subsystem 30 of the patient-side robotic telesurgery portal 10A. The provision of telepresence information allows for natural communication between the operating room with the patient and the robot 12 and the surgeon location with the surgeon and the robot control panel 14.

To ensure patient safety, the communication of surgical video, robot control feedback data and robot control data are prioritized and ensured. For example, in an implementation, the robotic telesurgery portals 10A, 10B will not allow the commencement of surgery until the presence of two redundance low-latency communication networks is verified. Additionally, in an implementation, if communication bandwidth becomes constrained, the communication of telepresence information may be restricted to ensure the communication of surgical video, robot control feedback data and robot control data. Additionally, in an implementation, variable encode/decode quality (dynamic compression levels) is used to vary data rates to ensure communication of essential surgical video, robot control feedback data and robot control data as necessitated by network conditions.

There is disclosed in the above description and the drawings a robot telesurgery portal system that fully and effectively overcomes the disadvantages associated with the prior art. However, it will be apparent that variations and modifications of the disclosed implementations may be made without departing from the principles of the invention. The presentation of the implementations herein is offered by way of example only and not limitation, with a true scope and spirit of the invention being indicated by the following claims.

What is claimed is:

1. A robotic telesurgery portal system comprising:
a first robotic telesurgery portal at a first location, the first robotic telesurgery portal being couplable to a surgical robot and configured to:
receive surgical video and robot control feedback data from the surgical robot;
synchronize the video and robot control feedback data from the surgical robot;
transmit the surgical video and robot control feedback data;
receive robot control information for the surgical robot; and
communicate robot control information to the surgical robot;
a second robotic telesurgery portal at a second location remote from the first location, the second robotic telesurgery portal being couplable to a surgical robot control console and configured to:
receive surgical video and robot control feedback data from the first robotic telesurgery portal;
communicate the received surgical video and robot control feedback data to the surgical robot control console;
receive robot control data from the surgical robot control console; and
transmit the received robot control data to the first robotic telesurgery portal.

2. The robotic telesurgery portal system of claim 1 wherein the first robotic telesurgery portal and the second robotic telesurgery portal are configured for multiple low-latency modes of secure and redundant communication.

3. The robotic telesurgery portal system of claim 2 wherein the at least one of the low-latency modes of communication is through a 5G cellular communication network.

4. The robotic telesurgery portal system of claim 2 wherein the first robotic telesurgery portal and the second robotic telesurgery portal are configured to prevent the commencement of a surgery until multiple modes of secure and redundant communication are verified.

5. The robotic telesurgery portal system of claim 1 wherein the first robotic telesurgery portal is further configured to synchronize surgical video and robot control feedback data prior to transmitting the surgical video and robot control feedback data.

6. The robotic telesurgery portal system of claim 1 wherein the first robotic telesurgery portal is further configured to encode the surgical video prior to transmitting the surgical video and robot control feedback data.

7. The robotic telesurgery portal system of claim 6 wherein the first robotic telesurgery portal is further configured to variably encode the surgical video depending on sensed communication bandwidth to ensure that sufficient surgical video and robot control feedback data is communicated to the second robotic telesurgery portal.

8. The robotic telesurgery portal system of claim 1 wherein each of the first robotic telesurgery
portal and the second robotic telesurgery portal further comprise:
a microphone;
a speaker;
a camera; and
a display;
wherein each of the first robotic telesurgery portal and the second robotic telesurgery portal are further configured to communicate telepresence data received from the microphones and cameras to the speakers and displays.

9. The robotic telesurgery portal system of claim 1 wherein a single robotic telesurgery portal is configurable as both the first robotic telesurgery portal and the second robotic telesurgery portal.
